(19) United States
(12) Patent Application Publication
(10) Pub. No.: US 2003/0026794 A1
(43) Pub. Date: Feb. 6, 2003

(54) Selective enzyme treatment of skin conditions

(76) Inventor: Howard Fein, Cincinnati, OH (US)

Correspondence Address: Beverly A. Lyman, Wood, Herron & Evans, L.L.P., 2700 Carew Tower, 441 Vine Street, Cincinnati, OH, 45202-2917, US

(21) Appl. No.: 09/919,102

(22) Filed: Jul. 31, 2001

(51) Int. Cl. A61K 38/54; A61K 38/46; A61K 38/48

(52) U.S. Cl. 424/94.2; 424/94.6; 424/94.63

(57) ABSTRACT

A method of treating skin conditions by providing compositions containing enzymes to selectively remove specific layers of skin. The depth of skin removed (that is, vertical surface treated) is regulated by the type and concentration of enzyme or enzymes in the composition. The surface area of skin removed (that is, radial surface treated) is regulated by the area of topical application. Conditions treatable by the method include, but are not limited to, age-related conditions such as lines and wrinkles, infections, pigmentary disorders, follicular disorders such as acne, and hyperkeratotic disorders such as warts. The inventive method and composition thus achieves the specificity and efficacy of more invasive methods such as surgery, while providing a composition that may be topically applied and is easy to use.

### FIELD OF THE INVENTION

The invention relates to the use of skin treatment compositions containing enzymes that selectively target one or more layers of skin.

### BACKGROUND OF THE INVENTION

The skin, the largest organ of the human body, is of interest from biological, medical and cosmetic points of view. Many products exist which purport to target one or more of these aspects of skin care. Confounding such treatment is the fact that conditions affecting the skin may be specific to the skin, such as psoriasis and atopic dermatitis, or may be manifestations of a general disease, such as general allergic reactions.

There are a variety of over-the-counter and prescription products that contain enzymes which are used for the purpose of skin care. Reported applications for these products include softening skin, treating skin conditions such as dryness, wrinkles, and acne, and/or removing devitalized or necrotic skin. In general, enzyme-containing products have been directed to conditions in which it is desirable to remove the upper layers of the skin. In this process, termed exfoliation, the skin that has been removed is eventually replaced with newly generated skin from surrounding structures.

One class of enzymes used in skin-related products is proteolytic enzymes, also called proteases or peptidases, which function to hydrolyze, or break down, proteins. Proteins known as adhesion molecules are present in the extracellular matrix of skin where they act to bind and anchor keratinocytes (cells that are present in the outermost layer of skin). When proteolytic enzymes are applied to the surface of skin, they hydrolyze adhesion molecules, resulting desirably in exfoliation of the upper skin layers. Generally, most of the topically applied enzyme-containing products contain proteolytic enzymes, and would thus be directed to applications that involve treating the epidermis (the outermost layer of skin).

There are several types of proteolytic enzymes. Papain, a proteolytic enzyme that is isolated from unripe papaya fruit, is effective in hydrolyzing esters and hydrophobic proteins, and has been used in various types of topical formulations. Papain-containing formulations may also contain other proteolytic enzymes such as bromelain, which is isolated from pineapples. The formulations may additionally contain protein denaturing chemicals such as urea, and/or other chemicals such as alpha hydroxyacid and salicylic acid, both of which lower the pH of the skin and cause exfoliation. As another example, proteolytic enzymes obtained from the bacterium Micrococcus sedentarius have been shown to degrade calluses in vitro. As still another example, U.S. Pat. No. 5,981,256 discloses the use of a recombinant stratum corneum chymotryptic enzyme (SCCE) for pharmaceutical and cosmetic skin care. SCCE, a serine proteolytic enzyme, is responsible for the degradation of desmosomal proteins, which play a role in the intercellular cohesion in the epidermis. SCCE activity results in cell shedding from the surface of the cornified surface layer. Hence, recombinant SCCE could be used for treating various skin disorders, especially those involving some type of keratinization disorder. While most of these products are available over-the-counter, some topical skin-care formulations that contain enzymes are available by prescription only. These include Accuzyme® (papain) and Granulex® (trypsin), whose application is limited for debridement of wounds.

There are, however, other skin conditions whose treatment involves more than just removal of the outer layers of the epidermis. Such conditions may affect, and hence require removal of, either deeper epidermal layers, full thickness epidermis, or even dermal and/or subcutaneous layers. Current treatment methods for these conditions usually consist of methods that destroy the skin at the treatment site. These methods include cryotherapy (freezing with liquid nitrogen), surgical removal, electrosurgery (tissue destruction with electricity), laser surgery (tissue destruction with light), burning, and chemical destruction with caustic agents such as trichloroacetic acid and phenol. Most of these methods are non-selective in terms of tissue destruction, and damage or destroy not only the affected site, but other healthy layers and surfaces of skin as well. These methods also incur pain, scarring, pigmentary alterations, delayed wound healing, and lesion recurrence.

Therefore, it would be desirable to combine the beneficial features of the above known skin treatment compositions and methods, such as the convenience and ease of use of a topically applied skin care product, with the and applicability of a more invasive method, to achieve selective destruction of affected layers of skin. Such a combination has not, until now, been disclosed.

### SUMMARY OF THE INVENTION

The invention is directed to a method for treating a patient having a condition involving the epidermal, and/or dermal, and/or subcutaneous layer of skin using a composition containing at least one enzyme that affects one or more particular layers of skin. In one embodiment, a physiologically acceptable formulation containing an effective amount of an enzyme that selectively affects one or more skin layers is administered topically to treat the condition. In another embodiment, the enzyme-containing formulation may be administered directly to an underlying skin layer. The enzyme, naturally occurring or synthetic, may belong to the class of oxidoreductases, transferases, hydrolases, lyases, isomerases, and/or ligases. The enzymes may be produced using recombinant techniques and may contain one or more modified amino acids while retaining a desired level of activity. Also included in the invention are enzymes that have the desired therapeutic activity but that are not currently classified in one of the above classifications.

The method may be used to treat a wide variety of conditions which include, but are not limited to, neoplasms, pigmentary disorders, infectious disorders, follicular disorders, hyperkeratotic disorders, inflammatory disorders, vascular disorders, aging disorders including photo-aging disorders, deposition disorders, connective tissue disorders, cutaneous cystic disorders, dandruff, seborrheic dermatitis, dry skin, corns, calluses, warts, freckles, acne, wrinkles, cysts, eczema, insect bites, lupus, varicose veins, tattoos, and/or scars.

The inventive composition contains at least one enzyme in a pharmaceutically acceptable formulation and an amount effective to remove the affected skin layer or layers. The amount of enzyme can range from about 1×10⁻⁹%^{w/v} to about 80%^{w/v} of the formulation, more particularly from about 1×10⁻⁵%^{w/v} to about 10%^{w/v} of the formulation.

The invention is additionally directed to a composition in a pharmaceutically acceptable formulation for selectively treating skin, and that contains one or more hydrolases in an amount ranging from about 1×10⁻⁹% by weight to about 80% by weight of the formulation, more particularly from about 1×10⁻⁵%^{w/v} to about 10%^{w/v} of the formulation. The hydrolase may be one or more of an esterase, a glycosidase, a peptidase such as collagenase, trypsin, papain, bromelain, elastase, etc., a phosphatase, a thiolase, a phospholipase, an amidase, a deaminase, and/or a ribonuclease.

Thus, in one embodiment, a method for treating a patient having a condition affecting at least one layer of skin is disclosed. A physiologically acceptable formulation containing at least one enzyme selective for a layer of skin affected by the condition is applied in situ in an amount and for a duration effective to remove the skin layer or layers and treat the condition.

In another embodiment, a composition contains at least one protease, such as trypsin, chymotrypsin, papain, bromelain, dispase, thermolysin, and/or V8 protease, at a concentration in the range of about 1×10⁻⁵%^{w/v} to about 10%^{w/v} in a pharmaceutically acceptable formulation to selectively effect an epidermal layer of skin. The composition may also contain other components, such as exfoliants, drugs, and/or cytotoxic agents.

In another embodiment, a composition for treating a patient having a skin condition affecting at least one epidermal, and/or dermal layer, and/or subcutaneous layer contains a hydrolase and is topically applied. The hydrolase is present in an amount in the range of about 1×10⁻⁵% by weight to about 10% by weight.

In other embodiments, methods are disclosed to treat skin by topically applying to an outermost layer of skin a composition containing a protease in a biologically acceptable formulation in an amount and formulation to selectively remove at least one epidermal layer containing a skin condition; by providing a composition containing a protease in a biologically acceptable formulation in an amount and formulation to selectively remove at least one dermal layer containing a skin condition; and by providing a composition containing a lipid hydrolyzing enzyme in a biologically acceptable formulation in an amount and formulation to selectively remove at least one subcutaneous layer containing a skin condition.

In another embodiment, a method to target skin treatment of an affected area is disclosed. A composition containing at least one enzyme in an amount and formulation effective to selectively target skin removal is provided to the affected area.

In another embodiment, a method of treating signs of aging in skin, such as xerosis, rhytids, loss of skin tone, acitinic damage, fine lines, and dyspigmentation, is disclosed. A protease-containing biologically acceptable composition is provided to an outermost layer of affected skin in an amount and formulation to selectively target the affected skin layers.

In another embodiment, a method for treating a condition affecting skin is disclosed by applying a composition to the affected skin, where the composition contains at least one enzyme at a concentration selective for regulating the depth of skin treatment, and is applied to an area of skin selective for regulating a radial surface of skin treatment.

These and other embodiments will be further appreciated from the following detailed description and examples.

### BRIEF DESCRIPTION OF THE DRAWINGS

FIG. 1 is a photograph of skin showing a seborrheic keratosis lesion.

FIG. 2 is a photograph of the skin lesion of FIG. 1 immediately after treatment with one embodiment of the inventive composition.

FIG. 3 is a photograph of the skin lesion of FIG. 1 three weeks post treatment.

FIG. 4 is a photograph of the skin lesion of FIG. 1 six months post treatment.

FIG. 5 is a photograph of a histologic section of skin with a seborrheic keratosis lesion.

FIG. 6 is a photograph of the histologic section of skin shown in FIG. 5 two hours after treatment with one embodiment of the inventive composition.

FIG. 7 is a photograph of the histologic section of skin shown in FIG. 5 twenty four hours after treatment.

### DETAILED DESCRIPTION OF THE INVENTION

The invention is directed to compositions and methods to selectively treat, alleviate, or prevent conditions in mammals that affect one or more layers of skin. For example, the compositions are chosen and formulated to selectively remove part or all of an epidermal layer of skin, and/or part or all of a dermal layer of skin. The compositions contain one or more enzymes from the oxidoreductase, transferase, lyase, isomerase, ligase, and hydrolase classes of enzymes to remove affected skin layers. In one embodiment, the composition and formulation selectively targets one or more epidermal layers. Alternatively, other areas of the skin, such as the deeper dermal and/or subcutaneous layers, may also be treated by increasing the concentration of an enzyme in the formulation, or by adding other enzymes or agents that will promote and/or demonstrate efficacy in deeper layers of the skin. For treatment of at least an epidermal layer, the composition may be applied topically and/or may be injected. For selective treatment of a dermal and/or subcutaneous layer, the composition may be administered directly to the desired layer, for example, by injection. In any route of administration, the composition and method avoids conventional destructive techniques such as cryotherapy or surgery, while providing comparable efficacy in treating numerous types of skin conditions.

In one embodiment, the enzyme is a protease or a mixture of proteases in a suitable formulation that is administered to the affected skin. In this embodiment, the inventive method involves one or more administrations of protease-containing formulations for alleviation, treatment, and/or prevention of skin conditions. Depending upon the layer or layers of skin to be treated, different proteases are used in the composition. For example, if only the epidermis is to be treated (that is, one or more epidermal layers), the composition may contain trypsin and/or papain. If only the dermis is to be treated, the composition may contain collagenase and/or elastase and may be administered directly into the dermis. If epidermal and dermal layers are to be treated, the composition may be applied topically and may contain trypsin and collagenase; trypsin and elastase; papain and collagenase; papain and elastase, trypsin, papain, and collagenase; trypsin, papain, and elastase; or trypsin, papain, collagenase, and elastase.

Skin conditions to be treated can be categorized into several groups, as the nature of each condition can be quite variable. One group of conditions is cosmetically undesirable, but is non-pathological, such as natural or artificial altered skin pigmentation. Another group of conditions is pathological but is readily treated or controlled, such as skin infections. Still another group of conditions is pathological and more invasive, such as neoplasms affecting the skin. Within this last group are neoplasms having various degrees of aggressiveness, ranging from the relatively non-aggressive basal cell carcinoma, to the aggressive malignant melanoma. The types and examples of each group are as follows.

One group is neoplastic disorders. These include, but are not limited to, actinic keratosis, apocrine gland neoplasm, Bowenoid papulosis, Bowen's disease, basal cell carcinoma, dermatofibroma, dermatosis papulosa nigrans, eccrine gland neoplasm, fibroepithelial polyp (skin tag), keratoacanthoma, Kaposi's sarcoma, lentigo maligna, lentigo maligna melanoma, melanoma, melanocytic nevus, neurofibroma, sebaceous gland neoplasm, sebaceous gland hyperplasia, seborrheic keratosis, squamous cell carcinoma, squamous cell carcinoma in situ, stucco keratoses, syringoma, and tricholemmoma.

A second group is pigmentary disorders. These include, but are not limited to, café au lait macule, chloasma, ephilide (freckle), lentigo (age spot), melasma, Mongolian spot, nevus of Ito, nevus of Ota, post-inflammatory hyperpigmentation, and post-inflammatory hypopigmentation.

A third group is infectious disorders. Infectious disorders encompass those caused by any infectious agent and include bacterial, parasitic, fungal, Mycobacteria, and viral pathogens. Infectious disorders include, but are not limited to, condyloma accuminatum (genital wart), dermatophytosis, verruca plana (flat wart), verruca vulgaris (common wart), molluscum contagiosum, onychomycosis, pediculosis capitis, pediculosis pubis, tinea, scabies, and tinea versicolor.

A fourth group is follicular disorders. These include, but are not limited to, acne vulgaris, acne keloidalis nuchae, comedone, folliculitis, hair follicle neoplasms, keratosis pilaris, pseudo-folliculitis barbae, and rosacea.

A fifth group is hyperkeratotic disorders. Hyperkeratosis is a pathologic disease state characterized by retention of keratinocytes; normal desquamation does not occur. These include, but are not limited to, acquired ichthyosis, acanthosis nigricans, epidermal nevus, hyperkeratotic dermatitis of palms and soles, ichthyosis, prurigo nodularis, ichthyosis vulgaris, lamellar ichthyosis, lichen simplex chronicus, palmoplantar keratoderma, xerosis (dry skin), X-linked ichthyosis and papillomatosis. This group also includes corns and calluses, both of which show thickening of the skin as a result of friction or pressure.

A sixth group is inflammatory disorders. These include, but are not limited to, atopic dermatitis, dermatitis, eczema, insect bites, lichen planus, lupus erythematosus, lymphomatoid papulosis, pityriasis lichenoides, psoriasis, sarcoid, scleroderma, seborrheic dermatitis, acne vulgaris and dandruff.

A seventh group is vascular disorders. These include, but are not limited to, cherry angioma, hemangioma, pigmented purpuric dermatoses, stasis dermatitis, telangectasia, varicose veins, vascular malformations and vascular neoplasms.

An eighth group is photo-aging disorders. These include, but are not limited to, actinic damage, photo-aging, photo-damage, poikiloderma, rhytid (wrinkles) and solar elastosis.

A ninth group is deposition disorders. These include, but are not limited to, amyloidosis, cutaneous mucinosis, cutis laxa, myxedema, tattoos and xanthomas.

A tenth group is connective tissue disorders. These include, but are not limited to, connective tissue nevus, dermal atrophy, fibrous papule, hypertrophic scars, keloids, lichen sclerosis, morphea, rhinophyma, cicatrix (scars) and striae.

An eleventh group is cutaneous cystic disorders. These include, but are not limited to, epidermal inclusion cyst and milium.

Advantageously, the inventive method can also encompass use of the enzyme composition as an exfoliant, that is, for use as a cosmetic peel. Such a cosmetic peel would be used to reduce the appearance of wrinkles, improve skin tone and texture, and/or reduce signs of skin aging. The method also encompasses use of the enzyme composition as a depilatory agent to remove excess hair or unwanted hair.

Enzymes are grouped into various classes, depending on the types of reactions catalyzed and their mechanism of action. Oxidoreductases catalyze oxidation/reduction reactions. Examples of oxidoreductases include dehydrogenases, oxidases, reductases, peroxidases, catalases, oxygenases and hydroxylases. Transferases transfer functional groups between donors and acceptors. Examples of transferases include transaldolase and transketolase; acyl, methyl, glucosyl, and phosphoryltransferases; kinases; and phosphomutases. Hydrolases are a special class of transferases in which the donor group is transferred to water. Examples of hydrolases include esterases, glycosidases, proteases, phosphatases, thiolases, lipases, phospholipases, amidases, deaminases, ceramidases, and nucleases. Lyases add or remove water, ammonia, and/or carbon dioxide. Examples of lyases include decarboxylases, aldolases, hydratases, dehydratases, synthases, and lyases. Isomerases catalyze isomerization reactions. Examples of isomerases include racemases, epimerases, isomerases, and some mutases. Ligases are involved in synthetic reactions. Examples of ligases include synthetases and carboxylases.

As used herein, the term enzyme encompasses both naturally occurring and synthetic enzymes. For example, an enzyme may be synthesized using recombinant techniques, as known to one skilled in the art. The term enzyme also encompasses enzymes that contain one or more modified amino acids, while still retaining desirable activity. Enzymes may also be modified to alter other properties such as, but not limited to, stability, target site affinity, substrate specificity, allosteric control, and required co-factors. In various embodiments, the enzyme may be isolated or may be added in a less than pure form, as long as its desired property is retained.

As previously described, hydrolases are used in one embodiment of the invention. Hydrolases are designated as Class 3 hydrolases according to the guidelines of the Nomenclature Committee of the International Union of Biochemistry and Molecular Biology, and can be subdivided into four groups depending upon the type of bond which is hydrolyzed by the enzymes. These groups are (1) Class 3.1, which act on ester bonds (esterases), examples of which include the lipid hydrolyzing enzymes such as lipases, phospholipases (phospholipase A₁, phospholipase A₂, phospholipase C, phospholipase D), sphingomylenases, cholesterol ester hydrolases, and ceramidases; (2) Class 3.2, which act on glycosidic bonds (glycosidases); (3) Class 3.3, which act on ether bonds; and (4) Class 3.4, which act on peptide bonds (proteases, also called peptidases), examples of which include trypsin, chymotrypsin, papain, collagenase, elastase, exopeptidases (carboxypeptidase A, carboxypeptidase B), aminopeptidases, endopeptidases, bromelain, chymotrypsin C, metridin, trypsin, thrombin, plasmin, enteropeptidase, alpha-lytic endopeptidase, prolyl oligopeptidase, brachyurin, plasma kallikrein, tissue kallikrein, pancreatic elastase, leukocyte elastase, chymase, cerevisin, hypodermin C, endopeptidase La, alpha-renin, leucyl endopeptidase, tryptase, kexin, subtilisin, oryzin, endopeptidase K, thermomycolin, thermitase, endopeptidase So, tissue plasminogen activator, pancreatic endopeptidase E, pancreatic elastase II, urine plasminogen activator, cathepsin B, ficain, chymopapain, asclepain, clostripain, streptopain, actinidain, cathepsin L, cathepsin H, calpain, cathepsin T, glycyl endopeptidase, cathepsin S, caricain, ananain, stem bromelain, fruit bromelain, legumain, histolysain, pepsin A, pepsin B, gastricsin, chymosin, cathepsin D, retropepsin, aspergillopepsin I, aspergillopepsin II, penicillopepsin, rhizopuspepsin, endothiapepsin, mucorpepsin, candidapepsin, saccharopepsin, rhodotorulapepsin, physaropepsin, acrocylindropepsin, polyporopepsin, pycnoporopepsin, scytalidopepsin A, scytalidopepsin B, xanthomonoapepsin, pseudomonapepsin, cathepsin E, atrolysin, microbial collagenase, leucolysin, interstitial collagenase, neprilysin, matrix metalloproteinases, dispase, thermolysin, and V8 protease. Although not included in the above classification, nucleases, such as deoxyribonuclease and ribonuclease, nucleosidases, and nucleoside phosphorylases are also encompassed by the invention.

In one embodiment of the invention, a composition contains one or more proteases. One function of proteases is to hydrolyze cellular adhesion proteins, which are found on either the surface of keratinocytes, the predominant cell type in the epidermis, or within the surrounding extracellular matrix. Examples of cellular adhesion proteins include desmogleins, lamins, integrins, bullous pemphigoid antigen 1 and 2, and others. They are responsible for adhesion of cells and maintenance of the structural integrity of the epidermis. Congenital absence of cellular adhesion proteins, such as in the disease epidermolysis bullosa, results in extreme epidermal fragility, which manifests clinically as blisters and erosions. Similarly, autoantibodies directed against these cell surface adhesion proteins, such as in bullous pemphigoid and pemphigus vulgaris, will also result in epidermal blisters and erosions. Proteases that hydrolyze cellular adhesion proteins have the potential to degrade both cell-surface proteins and those in the extracellular matrix. Without these inter- and extracellular attachments, the epidermis disintegrates in a process known as acantholysis. Thus, treating skin with proteases results in removal of one or more layers of skin. Moreover, cell adhesion proteins are located in the epidermis, which advantageously makes them readily accessible to topical enzymatic therapy.

In another embodiment of the invention, the composition contains one or more esterases, glycosidases, and/or nucleases. Esterases hydrolyze the various lipids that form cellular membranes of skin cells and the various lipids that form the epidermal intercellular lamellar lipid structures. Glycosidases hydrolyze the glycosidic bonds of skin proteins and lipids that have been naturally modified by glycosylation. Nucleases hydrolyze functional and non-functional nuclear material of viable and non-viable skin cells. These hydrolases and nucleosidases are used alone or in combinations as needed to selectively treat affected skin layers.

The concentration of the enzyme, or mixture of enzymes, in the composition is in the range from about 1×10⁻⁹%^{w/v} to about 80%^{w/v}, more specifically from about 1×10⁻⁵%^{w/v} to about ₁₀%^{w/v}. In one embodiment, the concentration of the enzyme, or mixture of enzymes, is in the range from about 1×10⁻⁴%^{w/v} to about 10%^{w/v}, more specifically from about 0.1%^{w/v} to about 3.0%^{w/v}. If the enzyme or enzymes is intended for topical administration to treat a condition with a single application, a concentration in the higher range would be useful. Such a formulation may contain an enzyme, or a mixture of enzymes, in the range from about 1%^{w/v} to about 5%^{w/v}. Alternatively, if the enzyme, or mixture of enzymes, is intended to achieve gradual tissue destruction, a concentration in the lower range would be useful, such as from about 1×10⁻²%^{w/v} to about 1.0%^{w/v}. Arbitrary concentration classifications for the enzyme, or mixture of enzymes, in the composition are from about 1×10⁻⁵%^{w/v} to about 1×10⁻³%^{w/v} and classified as a low strength composition, from about 1×10⁻²%^{w/v} to about 1%^{w/v} and classified as an intermediate strength composition, and from about 1%^{w/v} to about 10%^{w/v}, classified as a high strength composition.

A composition containing a lower concentration of the enzyme, or mixture of enzymes, would likely be available over the counter for self-administration by the patient. A composition containing a higher concentration of the enzyme, or mixture of enzymes, would likely be available only by prescription, or would be applied to the patient by a physician or other health care professional, rather than being self-administered.

The formulation may be administered at various intervals, depending upon factors such as the type of condition, the severity of the condition, the outcome desired, the concentration of the enzyme or mixture of enzymes, etc. The method may include application once a day, twice a day, once every other day, etc., as described in U.S. Pat. No. 5,981,256 which is expressly incorporated by reference herein in its entirety.

In one embodiment, the inventive method relates to use of an enzyme-containing composition suitable for topical application for the treatment of skin disorders or conditions affecting an epidermal layer of skin. This composition may include additional enzymes. A composition containing the protease papain could be present in a relatively low concentration and/or be formulated to affect only epidermal layers of skin, or could be present in a relatively higher concentration and/or formulated to affect some or all epidermal layers, or even dermal layers, of skin.

In alternative embodiments, the composition contains an enzyme such as collagenase that affects a dermal layer of skin, or an enzyme such as lipase that affects a subcutaneous layer of skin. These compositions may also contain additional enzymes, such as elastases.

In another embodiment, the inventive method relates to the use of a composition suitable for topical application as a cosmetic facial peel and that contains an enzyme or a mixture of enzymes for the treatment of skin conditions associated with aging, including photoaging. Such conditions include rhytides, loss of skin tone, fine lines, and dyspigmentation. Skin aging disorders may also be accelerated or complicated by photoaging disorders related to actinic damage, photo-damage, solar elastosis and poikiloderma. These conditions may involve layers of the skin ranging from only the few outer layers of the epidermis, to all epidermal layers, to epidermal and dermal layers. Hence, a variety of enzymes may be incorporated in the formulation to treat these conditions. The enzymes may include proteases, such as trypsin and papain, and ceramidases, either alone or in combination, for treatment of mainly the outer epidermal layers of the skin. These enzymes may also include phospholipases, lipases and collagenases to include treatment of the deeper epidermal layers and the dermis, depending upon the severity of the condition.

Any type of suitable, physiologically acceptable enzyme formulation may be used, as is known to one of skill in the art. Examples of such formulations include, but are not limited to, creams, ointments, lotions, emulsions, foams, aerosols, liniments, gels, solutions, suspensions, pastes, sticks, sprays, or soaps. Additionally, the inventive composition may be formulated so that it is encapsulated within a bead, sphere, capsule, microbead, microsphere, microcapsule, liposome, etc., as is known to one skilled in the art. Such formulations may advantageously release the composition over a period of time (time release formulations). The encapulated formulation may also be prepared as a concentrate or in a dry state or in a powder-like consistency to increase the shelf life of the enzyme preparation. Such formulations are diluted or reconstituted prior to administration and can be prepared using methods known to one skilled in the art.

The composition containing an enzyme or mixture of enzymes may also contain other compounds that have desirable therapeutic, cosmetic, and/or aesthetic properties, that either do not affect or only minimally affect the activity of the enzyme. These so-called co-additives may be used in any of the formulations that contain the enzyme. For example, gels or liquids may be useful in some instances in which rapid penetration is desired, such as when treatment occurs at certain intervals or in treatment of pediatric populations. A moisturizing cream base may be useful in other applications, such as in the treatment of xerosis (dry skin) and/or in the treatment of geriatric populations. One or more exfoliants, such as common chemical exfoliants including salicylic acid, lactic acid, alpha-hydroxy acids, beta-hydroxy acids, urea, and/or proteases, may also be added to the inventive composition. Other co-additives include divalent cation chelators such as ethylenediaminetetraacetic acid (EDTA) and ethylene glycol-bis (β-aminoethyl ether)-N,N,N¹,N¹-tetraacetic acid (EGTA), both of which sequester extracellular calcium, a necessary cofactor for the function of cellular adhesion molecules and hence which may be considered as exfoliants, since removal of calcium facilitates the activity of a protease in hydrolyzing the epidermal proteins. Other coadditives include exfoliants such as salicylic acid, lactic acid, alpha-hydroxy acids, beta-hydroxy acids, and/or urea, immunomodulating drugs such as glucocorticoids, tacrolimus, cyclosporin, interferon, ascomycin (SDZ ASM 981), imiquimod, or any of their respective derivatives and combinations thereof, cytotoxic agents such as podophyllin, podophylox, and/or cantharadin, and caustic agents.

Conventional skin destructive methods, such as topical application of liquid nitrogen and trichloroacetic acid, destroy tissue indiscriminately. The depth of tissue damage caused by these agents is a function of exposure time and/or concentration of the active agent. Conceivably, these conventional agents, if used in a sufficient concentration and for a sufficient exposure duration, would destroy not only full-thickness skin, but could produce damage extending into the deeper soft tissue and even bone. In contrast to conventional destructive agents, the inventive enzyme-containing compositions have the ability to produce selective tissue destruction limited to one or more layers of the skin, such as epidermis. For example, when the composition contains a protease and is topically applied, the enzyme may spread along the skin surface where it can interact with and destroy the epidermis only, or the epidermis and a portion of the dermis, or full-thickness skin. The skin layers affected can be changed by altering the particular enzyme(s) used. The depth of skin affected can be changed by altering the enzyme formulation, enzyme concentration, and/or exposure duration.

In the method, the composition may be applied at or adjacent to the affected site or sites. To limit the exposure to affected skin and to protect unaffected skin, or skin in which treatment is not desired, the composition may be formulated in a viscous material to form an ointment or other formulation in which inadvertent spread is prevented. Skin may also be protected from the composition through the use of physical barriers such as plastic wrap, petrolatum, petroleum jelly, etc. The composition may be formulated in a foam or gel, or within a device which could be cut precisely to the shape of the lesion. Alternatively, the composition may be applied at or adjacent to sites not yet affected, but sought to be treated for preventative or other reasons. The application may be performed in any manner that is suitable to the individual and/or the type of composition, and may additionally involve an application device. The composition may be applied directly or indirectly, such as by a dressing, bandage, covering, etc.

The duration and timing of treatment intervals and enzyme concentration in the composition can vary. Variables include the extent and type of lesion, the physical properties of the lesion, how long it takes for the lesion to be no longer visible, physician and patient preference, patient compliance, etc. As one example, a thick scaly lesion such as a verruca (wart) may require prolonged or repeated treatment relative to a flat non-scaly lesion. As another example, a physician administering the inventive composition may prefer a single treatment of a more concentrated dose than multiple treatments of less concentrated doses.

The treatment regimen may also vary, and the treatment time may be extended or shortened by varying the enzyme concentration and the formulation of the composition (e.g., a single application in contact with the lesion for fifteen minutes, or three applications in contact with the lesion for five minutes each). The treatment method may require short time intervals where the lesion, such as icthyosis, actinic keratosis, and the like, is located primarily in the epidermis, the outermost layer of skin. Longer times, and higher enzyme concentrations, are necessary to treat lesions such as cysts, connective tissue disorders and the like, which are located in the deeper layers of the skin and thus require the enzyme to penetrate deeper through a greater volume of tissue.

Depending upon the above parameters, healing of treated skin may take up to several weeks. After healing, treatment efficacy may be determined by visual inspection of the site by the physician and/or patient. One criterion of efficacy would be lack of visibility of the condition. For more intensive treatment, a biopsy or repeat biopsy made be performed, for example, to histologically verify destruction of a malignancy.

The following examples are directed to different embodiments of the invention and are illustrative, rather than limiting.

### EXAMPLE 1

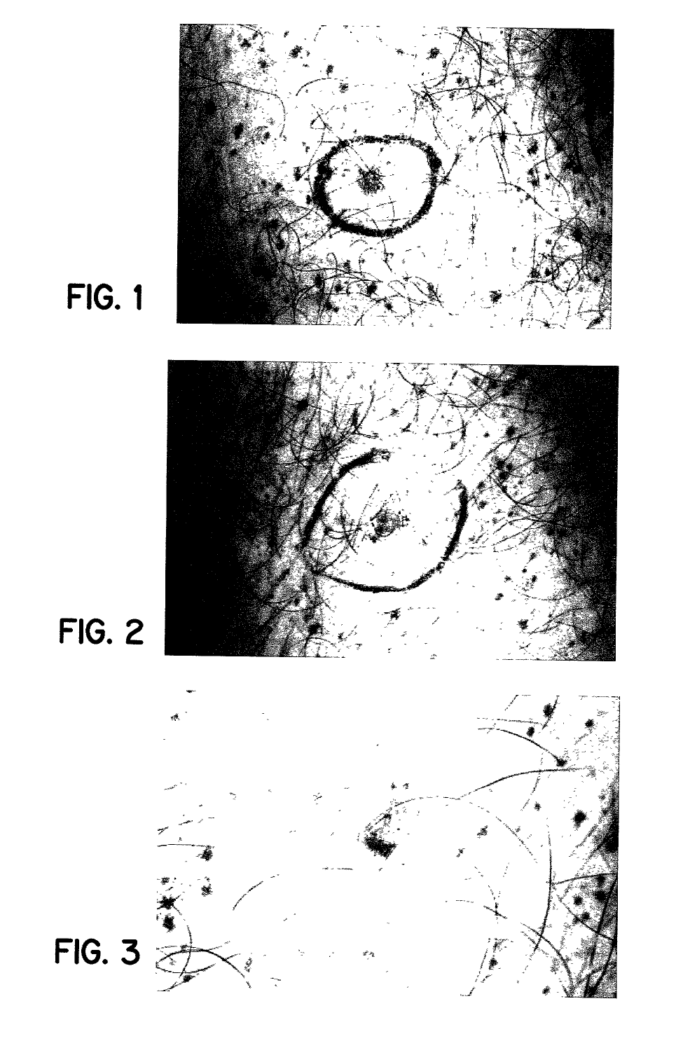

A patient was diagnosed as having a seborrheic keratosis, a benign neoplasm, on the left lower extremity as shown in FIG. 1. A composition of 2.5% trypsin, was formulated as a solution in phosphate buffered saline, pH 7.4 using methods known to one skilled in the art. The solution was applied topically to the site of the lesion, using a cotton-tipped applicator. The solution was reapplied, at intervals of three minutes, five additional times.

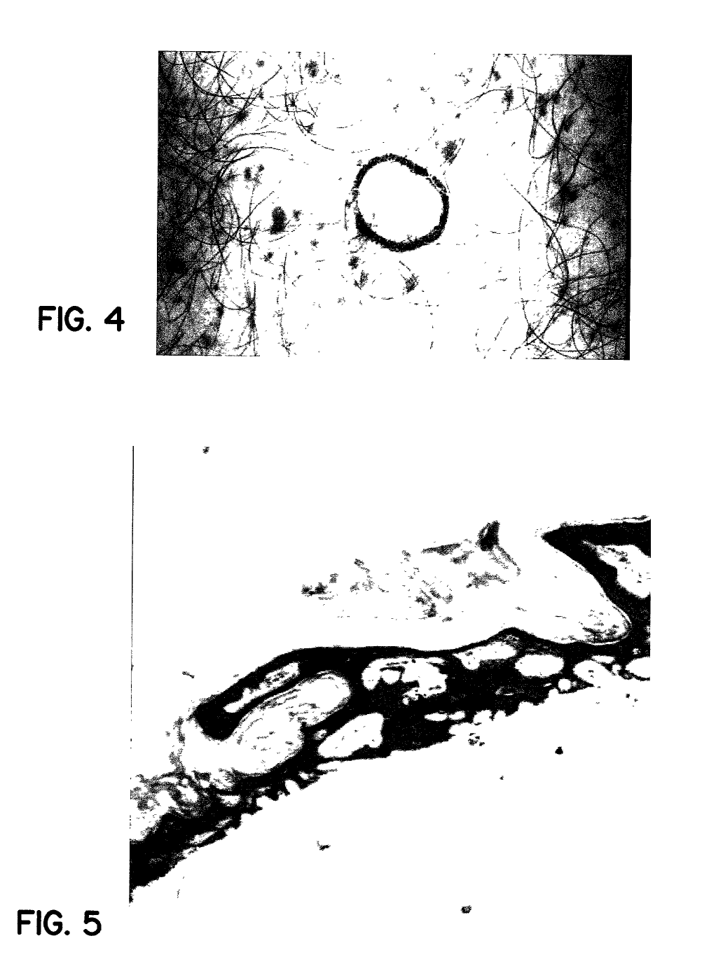

Immediately following the above-described treatment protocol, an erosion was present at the treatment site as shown in FIG. 2. The treated area was cleansed with soap and water and dressing was applied. At three weeks post-treatment, the lesion was completely eliminated without evidence of scarring or residual tissue as shown in FIG. 3. At six months post-treatment, there was no evidence of recurrence of the seborrheic keratosis as shown in FIG. 4.

### EXAMPLE 2

The patient is diagnosed as having an actinic keratosis behind the right ear. A composition of 2.5% trypsin and 2.5% bromelain is formulated as a solution in phosphate buffered saline, pH 7.4 using methods known to one skilled in the art. The solution is applied topically to the site of the lesion, using a cotton-tipped applicator. The composition is reapplied, at intervals of three minutes, five additional times.

### EXAMPLE 3

The patient is diagnosed as having a lentigo, a pigmentary disorder, on the right cheek. An enzyme composition is formulated and applied to the lesion as described in Example 1.

### EXAMPLE 4

The patient is diagnosed as having verruca plana, an infectious disorder, on the sole of the left foot. An enzyme composition is formulated and applied to the lesion as described in Example 2.

### EXAMPLE 5

The patient is diagnosed as having acne keloidalis nuchae, a follicular disorder, on the neck. A composition is formulated as a cream of 0.5% trypsin and 0.2% papain using methods known to one skilled in the art. The composition is applied topically to the lesion by spreading the cream in a thin layer. The composition is applied once every 24 hours until the condition disappears.

### EXAMPLE 6

The patient is diagnosed as having atopic dermatitis on the left wrist and right ankle. A composition is prepared as described in Example 5. The composition is applied to the lesions by spreading the cream in a thin layer.

### EXAMPLE 7

The patient is diagnosed as having pigmented purpuric dermatosis, a vascular disorder, on the lower extremities. A composition is formulated as an ointment of 0.2% trypsin and 0.1% papain using methods known to one skilled in the art. The composition is applied topically to the lesion as a thin film. The composition is reapplied every twelve hours.

### EXAMPLE 8

The patient is diagnosed as having actinic damage, a photoaging disorder, to the entire facial region. A composition is formulated and applied to the entire face as described in Example 5.

### EXAMPLE 9

The patient has a tattoo, a deposition within the skin, on the upper exterior portion of the right arm. A composition is formulated and applied as described in Example 1.

### EXAMPLE 10

The patient has hypertrophic scars, a connective tissue disorder, on the outside portion of the left thigh. A composition is formulated and applied as described in Example 1.

### EXAMPLE 11

The patient is diagnosed as having an epidermal inclusion cyst, a cutaneous cystic disorder, on the left cheek. A composition is formulated and applied as described in Example 1.

### EXAMPLE 12

The patient is diagnosed as having facial wrinkles and loss of skin tone as a result of aging. A composition is formulated and applied as described in Example 6.

### EXAMPLE 13

The patient is diagnosed as having ichthyosis vulgaris, a hyperkeratotic disorder, on the lower extremities. A composition is formulated as a cream of 0.001% trypsin using methods known to one skilled in the art. The composition is applied topically to the lesions by spreading the cream in a thin layer. The composition is applied twice every 24 hours until the condition resolves.

### EXAMPLE 14

To demonstrate the histological result of treatment by the invention, skin with seborrheic keratosis treated as described in FIG. 1 was removed from the patient by shave exision. A thin section of the skin was prepared and processed using standard methods for histologic examination. As shown in FIG. 5 (100× magnification) the seborrheic keratosis is intact and there is no other significant pathology.

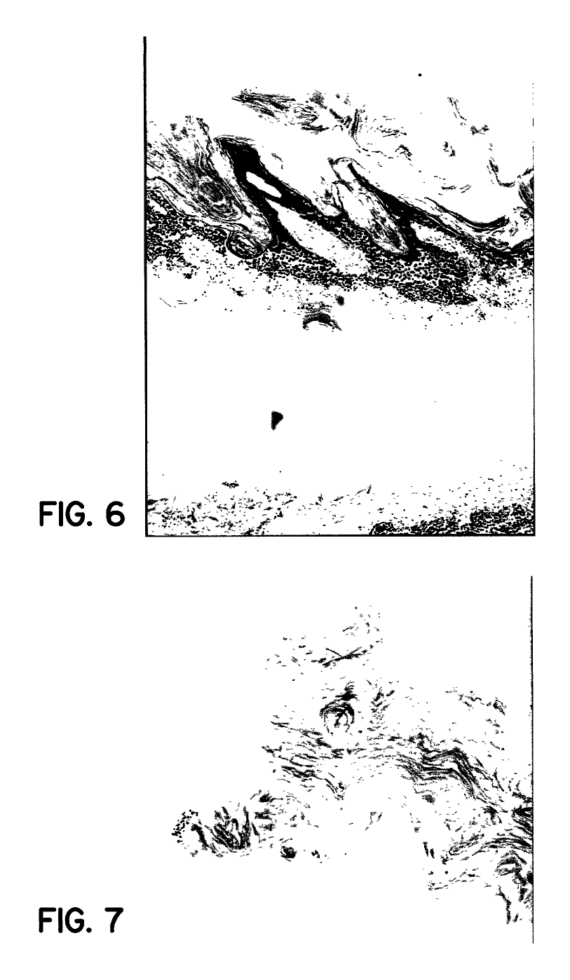

The remaining shaved skin section was placed in a petri dish. A saline solution containing 2.5%^{w/v} was applied to the surface of the lesion and incubated at 37° C. After two hours, another section of the skin was processed for histologic examination. As shown in FIG. 6 (100× magnification) there is near complete sub-epidermal detachment and marked epidermal acantholysis.

After 24 hours, incubation at 37° C. a section of the skin was processed for histologic examination. As shown in FIG. 7 (100× magnification) the epidermis was completely destroyed with only the stratum corneum remaining.

It should be understood that the embodiments of the present invention shown and described in the specification are only preferred embodiments of the inventor who is skilled in the art and are not limiting in any way. For example, the inventive composition and method may have veterinary applications for treatment of skin conditions in non-human animals. The dose, formulations, and other variables would be altered depending upon the particular species to be treated, as would be known by one skilled in the art. Therefore, various changes, modifications or alterations to these embodiments may be made or resorted to without departing from the spirit of the invention and the scope of the following claims.

What is claimed is:

1. A method for treating a patient having a condition affecting at least one layer of skin comprising administering in situ a physiologically acceptable formulation containing at least one enzyme selective for a layer of skin affected by said condition in an amount and for a duration effective to remove said layer and treat said condition.

2. The method of claim 1 wherein said layer of skin is selected from the group consisting of an epidermal layer, a dermal layer, a subcutaneous layer, and combinations thereof.

3. The method of claim 1 wherein said enzyme is a hydrolase.

4. The method of claim 3 wherein said hydrolase is selected from the group consisting of esterases, glycosidases, proteases, phosphatases, thiolases, phospholipases, amidases, ceramidases, lipases, deaminases, nucleases and combinations thereof.

5. The method of claim 1 wherein said enzyme is selected from the group consisting of trypsin, chymotrypsin, papain, bromelain, dispase, thermolysin, ceramidase, lipase, glycosidase, deoxyribonuclease, ribonuclease and combinations thereof to treat an epidermal layer.

6. The method of claim 1 wherein said enzyme is selected from the group consisting of collagenase, elastase, and combinations thereof to treat a dermal layer.

7. The method of claim 1 wherein said enzyme is a lipase to treat a subcutaneous layer.

8. The method of claim 1 where the condition is selected from the group consisting of neoplasms, pigmentary disorders, infectious disorders, follicular disorders, hyperkeratotic disorders, inflammatory disorders, vascular disorders, photo-aging disorders, deposition disorders, connective tissue disorders, and cutaneous cystic disorders.

9. The method of claim 1 wherein the enzyme is selected from the group consisting of trypsin, chymotrypsin, papain, collagenase, pepsin A, elastase, exopeptidases, aminopeptidases, endopeptidases, bromelain, chymotrypsin C, metridin, trypsin, thrombin, plasmin, enteropeptidase, alpha-lytic endopeptidase, prolyl oligopeptidase, brachyurin, plasma kallikrein, tissue kallikrein, pancreatic elastase, leukocyte elastase, chymase, cerevisin, hypodermin C, endopeptidase La, alpha-renin, leucyl endopeptidase, tryptase, kexin, subtilisin, oryzin, endopeptidase K, thermomycolin, thermitase, endopeptidase So, tissue plasminogen activator, pancreatic endopeptidase E, pancreatic elastase II, urine plasminogen activator, cathepsin B, facain, chymopapain, asclepain, clostripain, streptopain, actinidain, cathepsin L, cathepsin H, calpain, cathepsin T, glycyl endopeptidase, cathepsin S, caricain, ananain, stem bromelain, fruit bromelain, legumain, histolysain, pepsin B, gastricsin, chymosin, cathepsin D, retropepsin, aspergillopepsin I, aspergillopepsin II, penicillopepsin, rhizopuspepsin, endothiapepsin, mucorpepsin, candidapepsin, saccharopepsin, rhodotorulapepsin, physaropepsin, acrocylindropepsin, polyporopepsin, pycnoporopepsin, scytalidopepsin A, scytalidopepsin B, xanthomonoapepsin, pseudomonapepsin, cathepsin E, atrolysin, microbial collagenase, leucolysin, interstitial collagenase, neprilysin, matrix metalloproteinases dispase, thermolysin, V8 protease, ribonuclease, deoxyribonuclease. and combinations thereof.

10. The method of claim 1 wherein said administering is selected from the group consisting of topical application, injection, and combinations thereof.

11. A composition comprising at least one protease selected from the group consisting of trypsin, chymotrypsin, papain, bromelain, dispase, thermolysin, V8 protease, and combinations thereof at a concentration in the range of about 1×10⁻⁵%^{w/v} to about 10%^{w/v} in a pharmaceutically acceptable formulation to selectively effect an epidermal layer of skin.

12. The composition of claim 11 wherein said protease is selected from the group consisting of naturally occurring proteases, synthetic proteases, and combinations thereof.

13. The composition of claim 11 wherein the concentration of protease is in the range of about 1×10⁻⁴%^{w/v} to about 10%^{w/v}.

14. The composition of claim 11 wherein the concentration of protease is in the range of about 0.1%^{w/v} to about 3.0%^{w/v}.

15. The composition of claim 11 wherein the formulation is selected from the group consisting of creams, ointments, lotions, liniments, gels, solutions, suspensions, pastes, sticks, sprays, beads, spheres, microbeads, microspheres, liposomes, and combinations thereof.

16. The composition of claim 11 further containing a co-additive selected from the group consisting of exfoliants, immunomodulating drugs, cytotoxic drugs and combinations thereof.

17. The composition of claim 16 wherein said exfoliant is selected from the group consisting of ethylendiaminetetracetic acid (EDTA), ethylene glycol-bis (β-aminoethyl ether)-N,N,N¹,N¹-tetra acetic acid (EGTA), salicylic acid, lactic acid, alpha-hydroxy acids, beta-hydroxy acids, urea and combinations thereof.

18. The composition of claim 16 wherein said immunomodulating drug is selected from the group consisting of glucocorticoids, tacrolimus, cyclosporin, interferon, ascomycin (SDZ ASM 981), imiquimod, or any of their respective derivatives and their combinations thereof.

19. The composition of claim 16 wherein said cytotoxic agent is selected from the group consisting of podophyllin, podophylox, cantharadin, and combinations thereof.

20. A topically applied composition for treating a patient having a skin condition affecting at least one epidermal, and/or dermal layer, and/or subcutaneous layer comprising a hydrolase in a pharmaceutically acceptable formulation, said enzyme in an amount in the range of about 1×10⁻⁵% by weight to about 10% by weight of said formulation.

21. The composition of claim 20 wherein said enzyme is a hydrolase selected from the group consisting of esterases, glycosidases, proteases, phosphatases, thiolases, phospholipases, amidases, ceramidases, lipases, deaminases, nucleases and combinations thereof.

22. The composition of claim 21 wherein said hydrolase is selected from the group consisting of naturally occurring hydrolases, synthetic hydrolases, and combinations thereof.

23. The composition of claim 21 wherein said protease is selected from the group consisting of collagenases, trypsin, papain, bromelain, streptokinase, chymotrypsin, chymotrypsin C, elastases, exopeptidases, endopeptidases, metridin, thrombin, plasmin, enteropeptidase, alpha-lytic endopeptidase, prolyl oligopeptidase, brachyurin, plasma kallikrein, tissue kallikrein, pancreatic elastase, leukocyte elastase, chymase, cerevisin, hypodermin C, endopeptidase La, alpha-renin, leucyl endopeptidase, tryptase, kexin, subtilisin, oryzin, endopeptidase K, thermomycolin, thermitase, endopeptidase So, tissue plasminogen activator, pancreatic endopeptidase E, pancreatic elastase II, urine plasminogen activator, cathepsin B, papain, ficain, chymopapain, asclepain, clostripain, streptopain, actinidain, cathepsin L, cathepsin H, calpain, cathepsin T, glycyl endopeptidase, cathepsin S, caricain, ananain, stem bromelain, fruit bromelain, legumain, histolysain, pepsin A, pepsin B, gastricsin, chymosin, cathepsin D, retropepsin, aspergillopepsin I, aspergillopepsin II, penicillopepsin, rhizopuspepsin, endothiapepsin, mucorpepsin, candidapepsin, saccharopepsin, rhodotorulapepsin, physaropepsin, acrocylindropepsin, polyporopepsin, pycnoporopepsin, scytalidopepsin A, scytalidopepsin B, xanthomonoapepsin, pseudomonapepsin, cathepsin E, atrolysin, microbial collagenase, leucolysin, interstitial collagenase, neprilysin, matrix metalloproteinases, dispase, thermolysin, V8 protease and combinations thereof.

24. A method to treat skin comprising topically applying to an outermost layer of skin a composition containing a protease in a biologically acceptable formulation in an amount and formulation to selectively remove at least one epidermal layer containing a skin condition to thereby treat said skin.

25. The method of claim 24 wherein said protease is selected from the group consisting of trypsin, papain, bromelain, dispase, thermolysin, and combinations thereof.

26. A method to treat skin comprising providing a composition containing a protease in a biologically acceptable formulation in an amount and formulation to selectively remove at least one dermal layer containing a skin condition to thereby treat said skin.

27. The method of claim 26 wherein said composition is provided by an administration method selected from the group consisting of topical, injection, and combinations thereof.

28. The method of claim 26 wherein said protease is selected from the group consisting of collagenases, elastases, and combinations thereof.

29. A method to treat skin comprising providing a composition containing a lipid hydrolyzing enzyme in a biologically acceptable formulation in an amount and formulation to selectively remove at least one subcutaneous layer containing a skin condition to thereby treat said skin.

30. A method to target skin treatment of an affected area comprising providing to said affected area a composition containing at least one enzyme in an amount and formulation effective to selectively target skin removal in said affected area.

31. The method of claim 30 wherein said affected area is selected from the group consisting of epidermis, dermis, subcutaneous layer, and combinations thereof.

32. A method of treating aging in at least one layer of skin comprising providing a protease-containing biologically acceptable composition to an outermost layer of affected skin in an amount and formulation to selectively target said affected skin layers.

33. The method of claim 32 wherein said signs are selected from the group consisting of xerosis, rhytids, loss of skin tone, acitinic damage, fine lines, dyspigmentation, and combinations thereof.

34. A method for treating a condition affecting skin comprising applying a composition to the affected skin, the composition containing at least one enzyme at a concentration selective for regulating depth of skin treatment and applied to an area selective for regulating a radial surface of skin treatment.

35. The method of claim 34 wherein the enzyme is a protease.

36. The method of claim 34 wherein the composition is topically applied.

37. The method of claim 34 wherein the composition comprises a protease and is topically applied.

38. The method of claim 34 wherein the composition is applied one time.

39. The method of claim 34 wherein the enzyme is a protease to treat an epidermal layer.

40. The method of claim 34 wherein a relatively short duration of exposure and a relatively low enzyme concentration is used to treat a condition affecting an epidermal layer.

41. The method of claim 34 wherein a relatively long duration of exposure and a relatively high enzyme concentration is used to treat a condition affecting a dermal layer.
